(19) United States
(12) Patent Application Publication
(10) Pub. No.: US 2015/0045700 A1
(43) Pub. Date: Feb. 12, 2015

(54) PATIENT ACTIVITY MONITORING SYSTEMS AND ASSOCIATED METHODS

(71) Applicant: University of Washington through its Center for Commercialization, Seattle, WA (US)

(72) Inventors: Peter R. Cavanagh, Seattle, WA (US); Paul Manner, Seattle, WA (US); Andrea Hanson, Seattle, WA (US); Alexandre Bykov, Seattle, WA (US)

(21) Appl. No.: 14/456,848

(22) Filed: Aug. 11, 2014

Related U.S. Application Data
(60) Provisional application No. 61/864,131, filed on Aug. 9, 2013.
(60) Provisional application No. 61/942,507, filed on Feb. 20, 2014.

(51) Int. Cl. A61B 5/00; A61B 5/11

(52) U.S. Cl. CPC A61B 5/4528; A61B 5/1121; A61B 5/6802; A61B 5/749; A61B 5/742; A61B 5/7275
USPC 600/595

(57) ABSTRACT

Systems for monitoring patient activity and associated methods and systems are disclosed herein. In one embodiment, the system can be configured to receive data indicative of motion of a joint acquired by a sensor positioned proximate a patient's joint. The system can detect patterns in the acquired data, and match corresponding patient activities to the detected patterns. The system can generate a report listing the patient activities, which can be transmitted to the patient's medical practitioner.

### CROSS-REFERENCE TO RELATED APPLICATION(S)

This application claims the benefit of pending U.S. Provisional Application No. 61/864,131, filed Aug. 9, 2013, and pending U.S. Provisional Application No. 61/942,507, filed Feb. 20, 2014, both of which are incorporated herein by reference in their entireties.

### TECHNICAL FIELD

The present technology relates generally to systems and methods for monitoring a patient's physical activity. In particular, several embodiments are directed to systems configured to monitor movements of one or more of a patient's joints (e.g., a knee, an elbow, etc.) before or after a surgical procedure and/or an injury.

### BACKGROUND

Orthopedic surgical procedures performed on a joint (e.g., knee, elbow, etc.) often require significant recovery periods of time. During a typical post-surgical recovery period, a patient's progress may be monitored using only a subjective assessment of the patient's perception of success combined with only occasional visits (e.g., once per month) to a practitioner. Subjective assessments may include questionnaires asking questions such as, for example, “Are you satisfied with your progress?”; “Can you use stairs normally?” and/or “What level of pain are you experiencing?” The subjective answers to questionnaires may not be sufficient to form a complete assessment of a patient's post-surgery progress. Some patients, for example, may be incapable of determining on their own what constitutes satisfactory progress and/or a normal level of activity. In addition, pain tolerances can vary dramatically among patients. Furthermore, some patients may submit answers that reflect what the patients think their doctors want to hear, rather than providing a true evaluation of the joint performance.

### BRIEF DESCRIPTION OF THE DRAWINGS

FIG. 1A is an isometric side view of a patient monitoring device configured in accordance with embodiments of the present technology.

FIGS. 1B and 1C are partially schematic side views of the device of FIG. 1A shown on a leg of the patient after flexion and extension, respectively, of the leg.

FIG. 1D is a partially schematic side view of the device of FIG. 1A shown on an arm of patient.

FIG. 2 is a schematic view of patient activity monitoring system configured in accordance with an embodiment of the present technology.

FIG. 3 is a flow diagram of a method of monitoring patient activity configured in accordance with an embodiment of the present technology.

FIG. 4 is a sample report generated in accordance with an embodiment of the present technology.

FIG. 5 is a flow diagram of a method of analyzing data configured in accordance with an embodiment of the present technology.

FIG. 6A is a graph of data collected in accordance with an embodiment of the present technology. FIG. 6B is a graph of the data of FIG. 6A after processing in accordance with an embodiment of the present technology. FIG. 6C is a graph of a shapelet that can be compared to the data in FIG. 6A.

### DETAILED DESCRIPTION

The present technology relates generally to patient activity monitoring systems and associated methods. In one embodiment, for example, a patient activity monitoring device includes a first body and a second body configured to be positioned proximate a joint of a patient. A flexible, elongate member can extend from the first body to the second body. A first sensor or a plurality of sensors (e.g., one or more accelerometers) can be positioned in the first body and/or second body and can acquire data indicative of motion of the patient. A second sensor (e.g., a goniometer comprising one or more optical fibers) can extend through the elongate member from the first body toward the second body and acquire data indicative of a flexion and/or an extension of the patient's joint. A transmitter can be coupled to the first and second sensors and configured to wirelessly transmit (e.g., via Wi-Fi, Bluetooth, radio, etc.) data acquired from the first and second sensors to a computer. The computer may be housed in a mobile device that is configured to receive input (e.g., audio, video and/or touch input) from the patient. The computer can also be configured to transmit the acquired data from the first and second sensors and the input data to a remote server (e.g., via the Internet and/or another communications network). In some embodiments, for example, the device can further include a control surface configured to receive touch input from the user, one or more visual indicators and/or one or more microphones configured to receive audio input from the patient. In one embodiment, the device can include a battery configured to be rechargeable by movement of the first body relative to the second body. In another embodiment, the elongate member is configured to have a stiffness substantially less than a stiffness of the patient's joint. In some other embodiments, the first body, the second body and the elongate member are integrated into an article of clothing and/or a textile product (e.g., a fabric wrap, sleeve, etc.).

In another embodiment of the present technology, a system for monitoring a patient can include a receiver configured to receive data indicative of motion of a joint acquired by a sensor positioned on the patient proximate the joint. The system can also include memory configured to store the acquired data and executable instructions, and one or more processors configured to execute the instructions stored on the memory. The instructions can include instructions for detecting one or more patterns in the acquired data; determining one or more patient activities based on the one or more detected patterns; and/or automatically generating a report that includes a list of one or more of the patient activities occurring during a predetermined period of time. In one embodiment, the receiver, memory and the one or more processors are housed in a computer remote from the sensor (e.g., a remote server communicatively coupled to the receiver via the Internet and/or another communications network). In some embodiments, the system includes a mobile device coupled to the sensor via a first communication link and coupled to the receiver via a second communication link The mobile device can receive audio, video and touch input data from the patient, and can also transmit the data acquired by the sensor and the patient input data to the receiver via the second communication link. The generated report can include at least a portion of the patient input data received from the mobile device. In other embodiments, the system includes a transmitter configured to communicate with a medical information system via a communication link. The system can transmit the generated report to the medical information system. In some embodiments, the system can also trigger an alert to the patient's medical practitioner and/or an appointment for the patient in the medical information system. The triggering can be based on one or more of the patterns detected in the acquired data.

In yet another embodiment of the present technology, a method of assessing a function of a joint of a patient after a surgery performed on the joint includes receiving data from a sensor positionable proximate the patient's joint. The sensor can be configured to acquire data corresponding to an actuation of the patient's joint. The method also includes detecting one or more patterns in the acquired data, and determining one or more patient activities based on the one or more patterns detected in the acquired data. The method further includes automatically generating a report that includes, for example, a list and a duration of each of the one or more of the patient activities. In some embodiments, determining one or more patient activities can include comparing the one or more patterns detected in the acquired data with patterns in baseline data acquired from a different patient. In other embodiments, detecting one or more patterns in the acquired data can include reducing a number of dimensions in the acquired data from a first number of dimensions to a second, lower number of dimensions. In further embodiments, detecting one or more patterns can further include identifying shapelets in the data that are substantially mathematically characteristic of a patient activity. In another embodiment, the method can include transmitting the generated report to a medical information system. In yet another embodiment, the method can also include automatically scheduling an appointment based on one or more of the patterns detected in the acquired data.

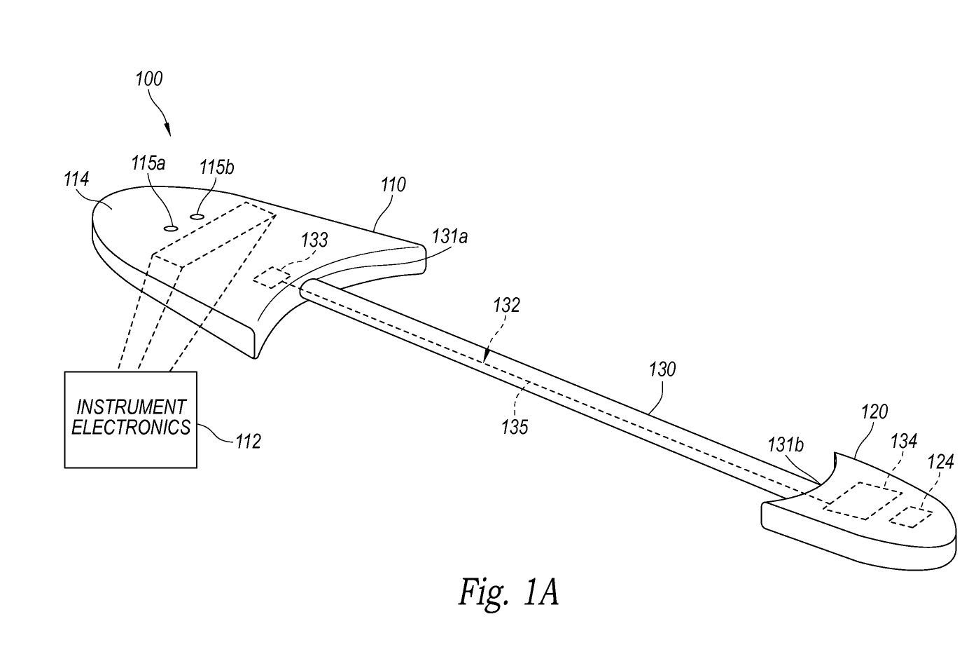

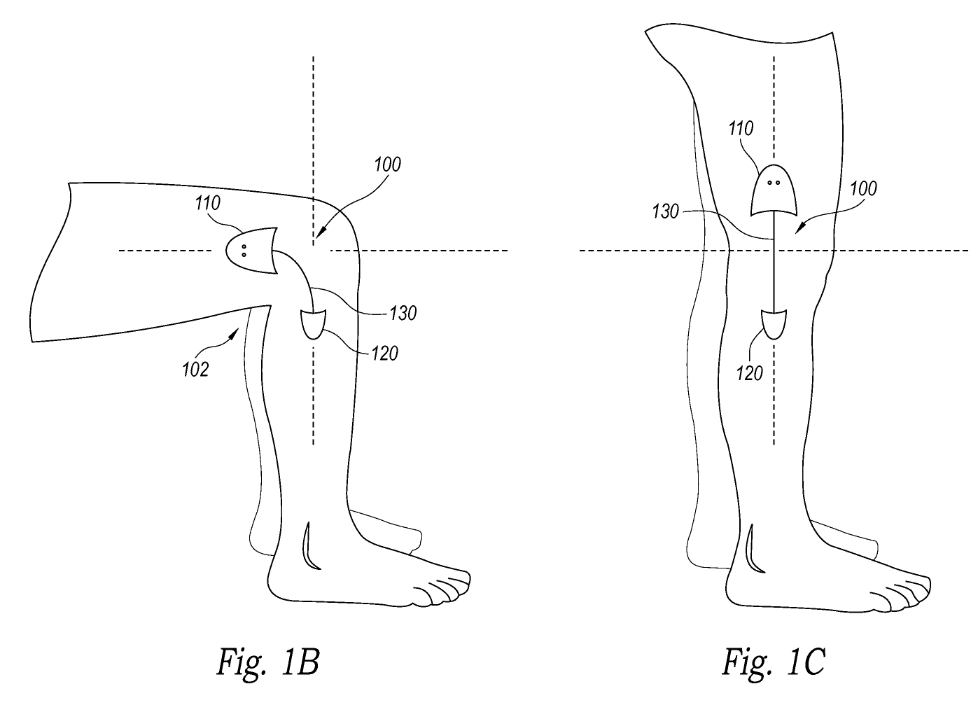

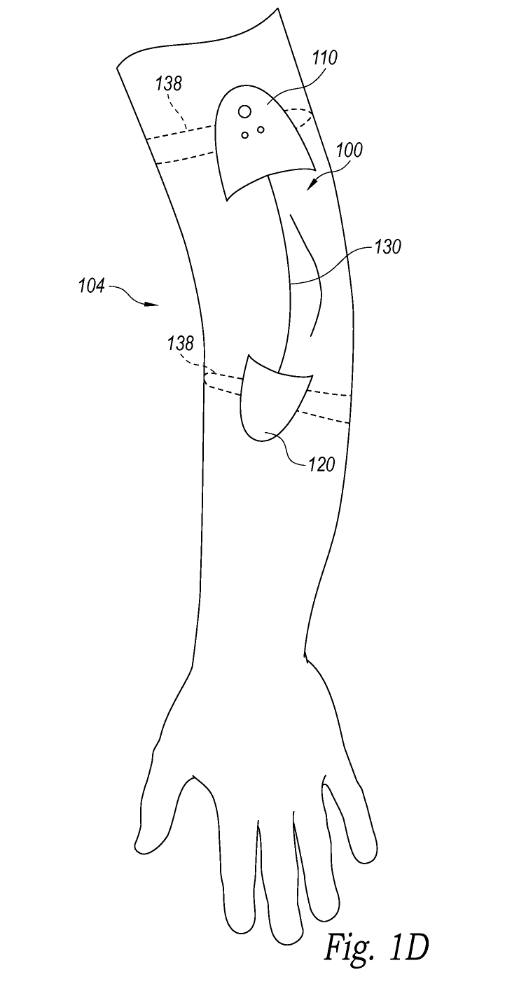

Certain specific details are set forth in the following description and in FIGS. 1-6C to provide a thorough understanding of various embodiments of the technology. Other details describing well-known structures and systems often associated with medical monitoring devices, data classification methods and systems thereof have not been set forth in the following technology to avoid unnecessarily obscuring the description of the various embodiments of the technology. A person of ordinary skill in the art, therefore, will accordingly understand that the technology may have other embodiments with additional elements, or the technology may have other embodiments without several of the features shown and described below with reference to FIGS. 1A-6C.

FIG. 1A is a side isometric view of a patient-monitoring device 100 configured in accordance with an embodiment of the present technology. The device 100 includes a first enclosure, housing or body 110 and a second enclosure, housing or body 120 that are removably attachable to a patient's body (e.g., near a joint such as a patient's knee, elbow, shoulder, ankle, hip, spine etc.). Instrument electronics 112 disposed in the body 110 can include, for example, one or more sensors (e.g., accelerometers, goniometers, etc.), a receiver and a transmitter coupled to the sensors, and one or more power sources (e.g., a battery). A control surface 114 (e.g., a button, a pad, a touch input, etc.) disposed on the first body 110 can be configured to receive input from the patient. A plurality of indicators 115 (identified separately in FIG. 1A as a first indicator 115a and a second indicator 115b) can provide feedback to the patient (e.g., indicating whether the device 100 is fully charged, monitoring patient activity, communicating with an external device, etc.). The second body 120 can include one or more electrical components 124 (shown as a single component in FIG. 1A for clarity), which can include for example, one or more sensors (e.g., accelerometers, goniometers, etc.), batteries, transmitters, receivers, processors, and/or memory devices.

A coupling member 130 extends from a first end portion 131a attached to the first body 110 toward a second end portion 131b attached to the second body 120. The coupling member 130 can be made of, for example, rubber, plastic, metal and/or another suitable flexible and/or bendable material. In the illustrated embodiment of FIG. 1A, the coupling member 130 is shown as an elongate member. In other embodiments, however, the coupling member 130 can have any suitable shape (e.g., an arc). Moreover, the illustrated embodiment, a single coupling member 130 is shown. In other embodiments, however, additional coupling members may be implemented in the device 100. In further embodiments, the coupling member 130 may comprise a plurality of articulating elements (e.g., a chain). In some embodiments, the coupling member 130 may have a stiffness much lower than a stiffness of a human joint such that the device 100 does not restrain movement of a joint (e.g., a knee or elbow) near which the device 100 is positioned and/or monitoring. In certain embodiments, the device 100 the coupling member 130 may be replaced by, for example, one or more wires or cables (e.g., one or more electrical wires, optical fibers, etc.).

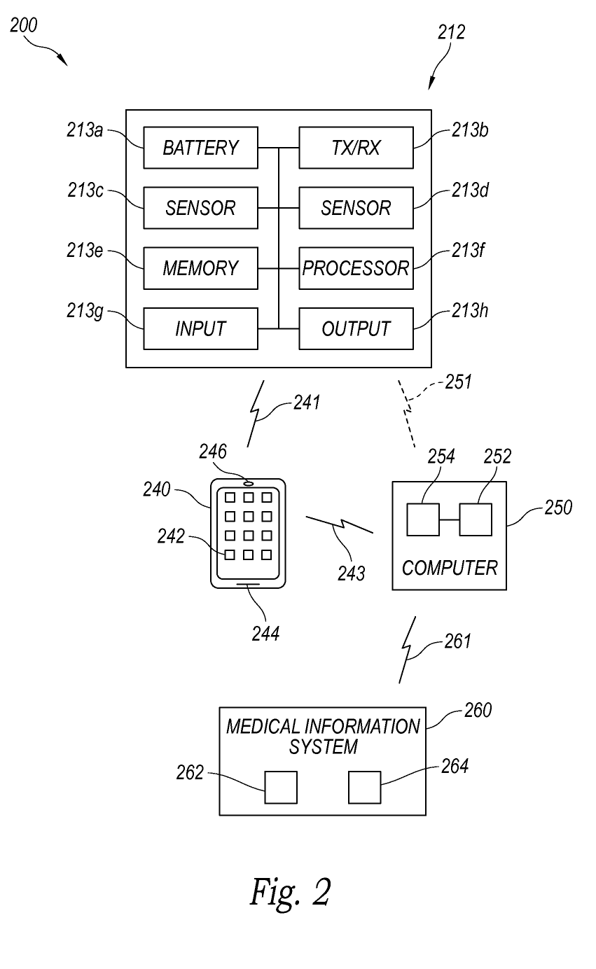

An angle sensor 132 (e.g., a goniometer) extends through the coupling member 130. A first end portion 133 of the angle sensor 132 is disposed in the first body 110, and a second end portion 134 of the angle sensor 132 is disposed in the second body 120. One or more cables 135 extend through the coupling member 130 from the first end portion 133 toward the second end portion 134. The cables 135 can include, for example, one or more electrical cables (e.g., resisitive and/or capacitive sensors) and/or one or more optical fibers. During movement of the patient's joint (e.g., flexion and/or extension of the patient's joint), the coupling member 130 bends and an angle between the first body 110 and the second body 120 accordingly changes. The angle sensor 132 can determine a change in angle between the first body 110 and the second body 120. If the cables 135 include electrical cables, the angle can be determined by measuring, for example, an increase or decrease in the electrical resistance of the cables 135. If the cables include optical fibers, the angle can be determined by measuring, for example, an increase or decrease in an amount of light transmitted through the cables 135. As explained in further detail with reference to FIG. 2, data acquired by the angle sensor 132 can be stored on memory in and/or on the electronics 112.

FIGS. 1B and 1C are partially schematic side views of the device 100 shown on a leg of the patient after flexion and extension, respectively, of a knee 102 of the patient's leg. FIG. 1D is a partially schematic side view of the device 100 shown on an arm of patient proximate an elbow 104 of the patient's arm. Referring to FIGS. 1A-1D together, the first body 110 and the second body 120 are configured to be positioned at least proximate a joint (e.g., a knee, wrist, elbow, shoulder, hip, ankle, spine, etc.) on the patient's body. In the illustrated embodiment of FIGS. 1B and 1C example, the first body 110 is positioned above the knee 102 (e.g., on a thigh adjacent an upper portion of the knee 102) and the second body 120 is positioned below the knee 102 (e.g., on an upper portion of the patient's shin adjacent the knee 102). In other embodiments, however, the first body 110 and the second body 120 can be positioned in any suitable arrangement proximate any joint of a patient's body. Moreover, in some embodiments the first body 110 and/or the second body 120 can be removably attached to the patient's body with a medical adhesive (e.g., hydrocolloidal adhesives, acrylic adhesive, a pressure sensitive adhesive, etc.) and/or medical tape. In other embodiments, however, any suitable material or device for positioning the device 100 at least proximate a joint of a patient may be used. In the illustrated embodiment of FIG. 1D, for example, the first body 110 and the second body 120 are attached to the patient's body proximate the patient's elbow using corresponding straps 138 (e.g., Velcro straps). In certain embodiments (not shown), the first body 110, the second body 120 and/or the coupling member 130 can be integrated, for example, into a wearable sleeve, a garment to be worn on the patient's body and/or in a prosthesis surgically implanted in the patient's body.

FIG. 2 and the following discussion provide a brief, general description of a suitable environment in which the technology may be implemented. Although not required, aspects of the technology are described in the general context of computer-executable instructions, such as routines executed by a general-purpose computer (e.g., a computer integrated within and/or communicatively coupled to the device 100 of FIGS. 1A-1D). Aspects of the technology can be embodied in a special purpose computer or data processor that is specifically programmed, configured, or constructed to perform one or more of the computer-executable instructions explained in detail herein. Aspects of the technology can also be practiced in distributed computing environments where tasks or modules are performed by remote processing devices, which are linked through a communication network (e.g., a wireless communication network, a wired communication network, a cellular communication network, the Internet, a hospital information network, etc.). In a distributed computing environment, program modules may be located in both local and remote memory storage devices.

Computer-implemented instructions, data structures, screen displays, and other data under aspects of the technology may be stored or distributed on computer-readable storage media, including magnetically or optically readable computer disks, as microcode on semiconductor memory, nanotechnology memory, organic or optical memory, or other portable and/or non-transitory data storage media. In other embodiments, aspects of the technology may be distributed over the Internet or over other networks (e.g., one or more HIPAA-compliant wired and/or wireless networks), on a propagated signal on a propagation medium (e.g., an electromagnetic wave(s), a sound wave, etc.) over a period of time, or may be provided on any analog or digital network (packet switched, circuit switched, or other scheme).

FIG. 2 is a schematic block diagram of a patient activity monitoring system 200. The system 200 includes electronics 212 (e.g., the electronics 112 shown in FIG. 1A) communicatively coupled to a mobile device 240 via a first communication link 241 (e.g., a wire, a wireless communication link, etc.). A second communication link 243 (e.g., a wireless communication link or another suitable communication network) communicatively couples the mobile device to a computer 250 (e.g., a computer such as a desktop computer, a laptop computer, a mobile device, a tablet, one or more servers, etc.). In some embodiments, the electronics 212 can be communicatively coupled directly to the computer 250 via a third communication link 251 (e.g., a wireless communication link connected to the Internet or another suitable communication network). A fourth communication link 261 (e.g., the Internet and/or another suitable communication network) couples the computer 250 to a medical information system 260 [e.g., a hospital information system that includes the patient's electronic medical record (EMR)]. As described in further detail below, the computer 250 can receive data from one or more sensors on the electronics 212, analyze the received data and generate a report that can be delivered to a medical practitioner monitoring the patient after a joint surgery and/or injury.

The electronics 212 can be incorporated, for example, in and/or on a sensor device (e.g., the device 100 of FIGS. 1A-1D) positionable on or proximate a joint of a patient before or after a surgical operation is performed on the joint. A battery 213a can provide electrical power to components of the electronics 212 and/or other components of the sensor device. In one embodiment, the battery 213a can be configured to be recharged via movement of the sensor device (e.g., movement of the device 100 of FIGS. 1A-1D). In other embodiments, however, the battery 213a can be rechargeable via a power cable, inductive charging and/or another suitable recharging method. A transmit/receive unit 213b can include a transmitter and receiver configured to wirelessly transmit data from the electronics 212 to external devices (e.g., mobile device, servers, cloud storage, etc.). A first sensor component 213c and a second sensor component 213d (e.g., sensors such as accelerometers, magnetometers, gyroscopes, goniometers, temperature sensors, blood pressure sensors, electrocardiograph sensors, global positioning system receivers, altimeters, etc.) can detect and/or acquire data indicative of motion of a patient, indicative of a flexion and/or extension of a patient's joint, and/or indicative of one or more other measurement parameters (e.g., blood pressure, heart rate, temperature, patient location, blood flow, etc.) In some embodiments, the electronics 212 can include one or more additional sensors (not shown in FIG. 2 for clarity). In other embodiments, however, the electronics 212 may include a single sensor component (e.g., the first sensor component 213c).

Memory 213e (e.g., computer-readable storage media) can store data acquired by the first and second sensor components 213c and 213d. The memory 213e can also store executable instructions that can be executed by one or more processors 213f. An input component 213g (e.g., a touch input, audio input, video input, etc.) can receive input from the patient and/or a medical practitioner (e.g., a doctor, a nurse, etc.). An output 213h [e.g., an audio output (e.g., a speaker), a video output (e.g., a display, a touchscreen, etc.), LED indicators (e.g., the first indicator 115a and the second indicator 115b shown in FIG. 1A), etc.] can provide the patient and/or the practitioner information about the operation or monitoring of the sensor device. The first communication link 241 (e.g., a wire, radio transmission, Wi-Fi, Bluetooth, and/or another suitable wireless transmission standard) communicatively couples the electronics 212 to the mobile device 240.

The mobile device 240 (e.g., a cellular phone, a smartphone, tablet, a personal digital assistant (PDA), a laptop and/or another suitable portable electronic device) includes a user interface 242 (e.g., a touch screen interface), an audio input 244 (e.g., one or more microphones), an audio output 246 (e.g., one or more speakers), and a camera 248. The mobile device 240 can receive information from the electronics 212 collected during patient activity (e.g., data acquired by the first and second sensor components 213c and 213d). The mobile device 240 can also include, for example, an executable application configured to gather subjective input and/or feedback from the patient. The patient can provide feedback via the application that includes, for example, touch input (e.g., via the user interface 242), audio input (e.g., via the audio input 244) and/or video input (e.g., an image or video of a joint being monitored captured via the camera 248). The feedback data and/or other data received from the electronics 212 can be transmitted to the computer 250 via the second communication link 243 (e.g., a wire and/or a wireless communication network connected to the Internet and/or another suitable communication network).

The computer 250 (e.g., a desktop computer, a laptop computer, a portable computing device, one or more servers, one or more cloud computers, etc.) can include, for example, one or more processors 252 coupled to memory 254 (e.g., one or more computer storage media configured to store data, executable instructions, etc.). As explained in further detail below, the computer 250 can be configured to receive data from the electronics 212 (e.g., via the third communication link 251) and/or directly from the mobile device 240 (e.g., via the second communication link 243). The computer 250 can process the received data to generate one or more reports that can be transmitted via the fourth communication link 261 (e.g., a wire and/or a wireless communication network connected to the Internet and/or another suitable communication network) to the medical information system 260.

The medical information system 260 includes a first database 262 (e.g., an EMR database) and a second database 264 (e.g., a database configured to store medical and/or hospital information such as scheduling, patient appointments, billing information, etc.). The patient's doctor and/or another medical practitioner monitoring the patient's activity can access the report generated by the computer 250 via the medical information system 260. In some embodiments, the computer 250 and/or the medical information system 260 can be configured to automatically schedule an appointment for the patient based on information contained in a report generated by the computer 250. For example, the report may include subjective feedback and/or patient activity data indicative of improper healing of the patient's joint after surgery. The computer 250 and/or the medical information system 260 can automatically add a new appointment in a scheduling database (e.g., stored in the second database 264). In another embodiment, the computer can alert the health care team regarding important information in either the patient's response to questions or in the measured data.

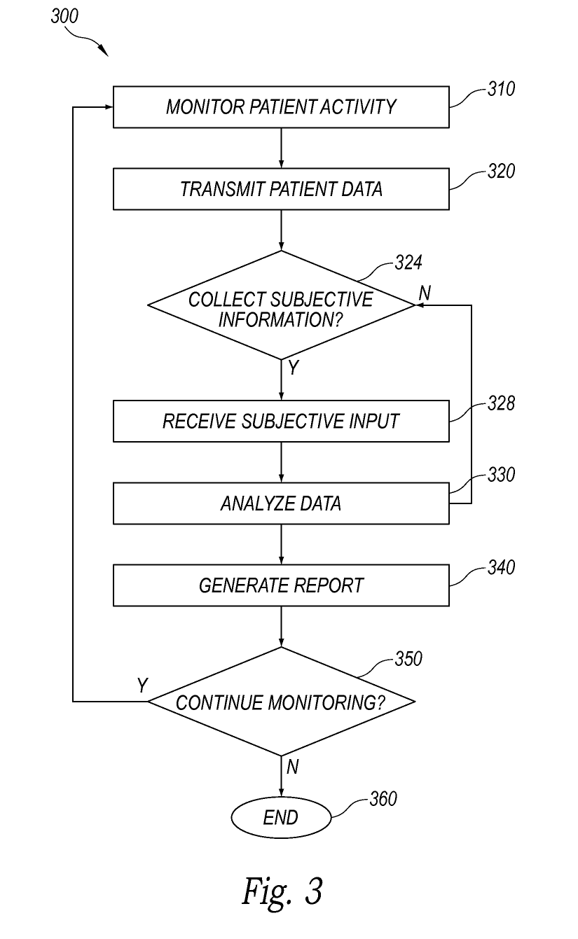

FIG. 3 is a flow diagram of a process 300 configured in accordance with the present technology. In one embodiment, the process 300 can comprise instructions stored, for example, on the memory 254 of the computer 250 (FIG. 2) and executed by the processor 252. In some embodiments, however, the process 300 can be executed by electronics (e.g., electronics 112 of FIG. 1A and/or the electronics 212 of FIG. 2) stored on a sensor device (e.g., the device 100 of FIGS. 1A-1D) proximate a patient's joint (e.g., a knee, elbow, ankle, etc.). In other embodiments, the process 300 can be stored and executed on a mobile device (e.g., the mobile device 240 of FIG. 2) communicatively coupled to the sensor device.

At step 310, the process 300 monitors patient activity, for example, by receiving information from the device 100 (e.g., from the first and second sensor components 213c and 213d shown in FIG. 2 and/or one or more other sensor components). The process 300 can use the information to compute patient information such as, for example, total active time of the patient, a distance traveled by the patient and/or a number of steps taken by the patient during a predetermined period of time (e.g., a day, a week, etc.) and/or a period of time during which the patient performs one or more activities. At step 320, patient data is transmitted, for example, from the device 100 to the computer 250 (FIG. 2) via a communication link (e.g., the first communication link 241, second communication link 243 and/or the third communication link 251 of FIG. 2).

At step 324, the process 300 determines whether subjective information is to be collected from the patient. If subjective information is to be collected from patient, the process 300 continues onto step 328 where it receives touch, audio, photographic and/or video input from the patient, for example, via the mobile device 240 of FIG. 2. The subjective input can include, for example, a photograph of the joint, a subjective indication of pain (e.g., a patient's subjective indication of pain on a scale from 1 to 10) and/or audio feedback from the patient during a movement of the joint.

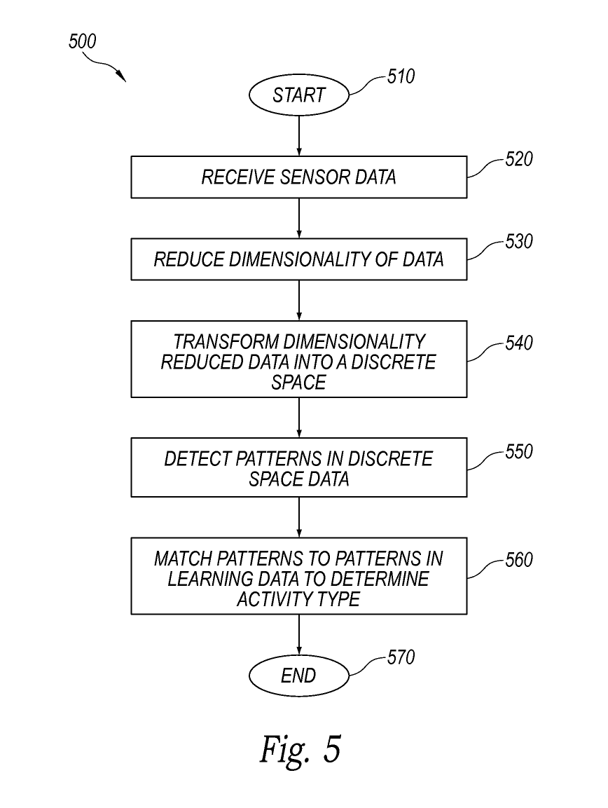

At step 330, the process 300 receives and analyzes data acquired by one or more sensors (e.g., the first and second sensor components 213c and 213d shown in FIG. 2). The process 300 analyzes the acquired data to determine, for example, a range of motion of the joint and/or one or more types of patient activity occurring during a measurement period (e.g., 1 hour, 1 day, etc.). The process 300 can calculate a range of motion of the joint using, for example, a total range traveled by the joint (e.g., a number of degrees or radians per day or another period of time) and/or extrema of one or more joint motions (e.g., maximum flexion, extension, abduction, adduction, internal rotation, external rotation, valgus, varus, etc.) The process 300 can also analyze every individual joint motion that occurs during a predetermined measurement period. For example, the process 300 can recognize one or more occurrences of a joint flexion movement to determine an extent of movement of the joint between and/or during flexion and extension of the joint. The process 300 can group movements into one or more data distributions that include a number of movements that occurred during a measurement period and/or a portion thereof. The process 300 can further calculate statistics of the distributions such as, for example, mean, mode, standard deviation, variance, inter-quartile ranges, kurtosis and/or skewness of the data distribution. As described in further detail below with reference to FIG. 5, the process 300 can also analyze sensor data to determine one or more activity types that the patient experienced during the measurement period. For example, the process 330 can analyze the sensor data and determine patterns in the data corresponding to periods of time when the patient was lying down, sitting, standing, walking, taking stairs, exercising, biking, etc.

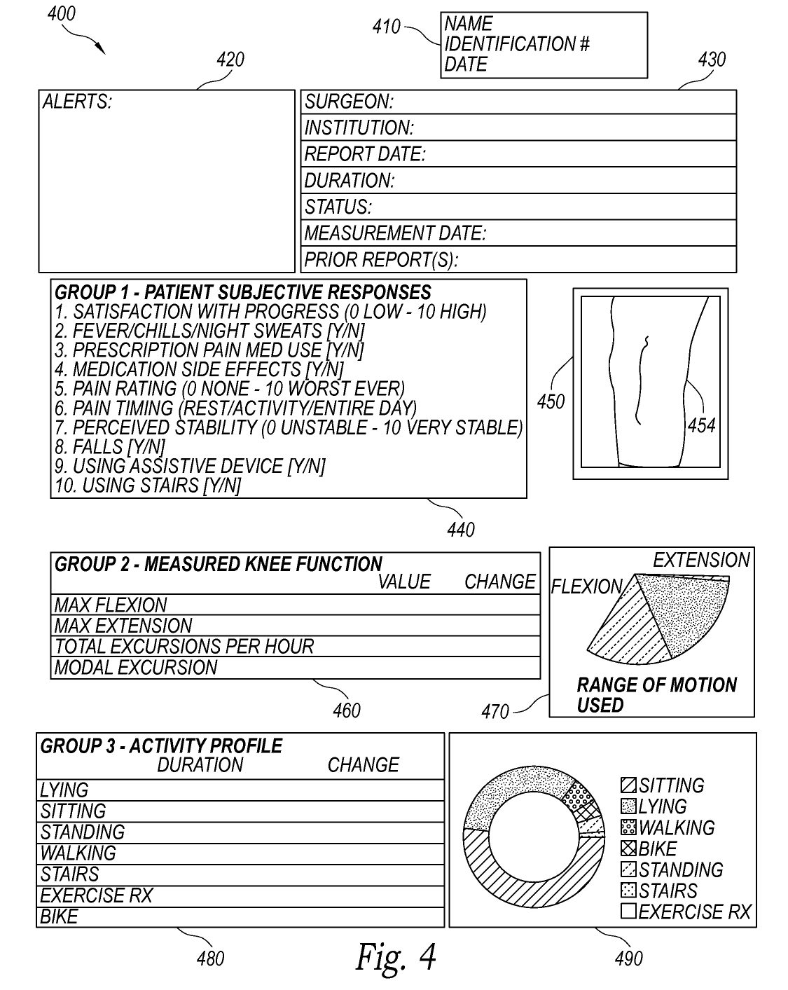

The process 300 at step 340 generates a report based on the analyzed data. As discussed in more detail below with reference to FIG. 4, the generated report can include, for example, patient subjective input from step 328 and/or an analysis of the data from step 330 along with patient identification information and/or one or more images received from the patient. The process 300 can transmit the report to the patient's medical practitioner (e.g., via the medical information system 260 of FIG. 2) to provide substantially immediate feedback of joint progress. In one embodiment, the process 300 may only report changes in the patient's joint progress since one or more previous reports. In some embodiments, the process 300 generates alerts to the medical practitioner when results of joint measurement parameters or activity recognition are outside normal limits for the reference group to which the patient belongs (e.g., a reference group of patients selected on a basis of similar body weight, height, sex, time from surgery, age, etc.). The process 300 can also deliver alerts that include, for example, a request for special priority handling, which may increase a likelihood that the patient's condition receives attention from the patient's medical practitioner. The process 300 can also automatically trigger a scheduling of a new appointment and/or the cancellation of a prior appointment based on one or more items in the report.

The report generated in step 340 can be used, for example, by the patient's medical practitioner and/or the patient to evaluate progress of the patient's joint at a predetermined time after a surgical operation performed on the joint. Embodiments of the present technology are expected to provide an advantage of providing the medical practitioner information about the actual activity profile of the patient rather than forcing the practitioner to rely, for example, solely on patient self-reported information (e.g., input received at step 328). Information in the report generated in step 340 can also allow medical practitioners to determine much sooner than certain prior art methods that additional treatment is necessary (e.g., physical therapy, mobilization of the joint under anesthesia, etc.). Moreover, the report can also provide information to the medical practitioner whether the patient is performing, for example, one or more prescribed therapeutic exercises. The report can also assist the medical practitioner in determining skills to be emphasized during therapeutic exercises based on the activities detected during step 330. At step 350, the process 300 determines whether to return to step 310 for additional monitor or whether to end at step 360.

FIG. 4 is a sample report 400 generated, for example, by the process 300 (FIG. 3) at step 340. FIG. 4 includes an identification field 410, which can list, for example, a patient's name, identification number, and the date that the report was generated. Field 420 can include one or more alerts that have been generated based on an analysis of the data and/or subjective input. The alerts can be generated, for example, by the process 300 during step 340 (FIG. 3). A third field 430 can include information, for example, about the patient's surgery, where the patient's surgery was performed, the name of one or more doctors who performed the surgery, the amount of time since the surgery occurred, the date that the measurement occurred, and one or more dates of previous reports. A fourth field 440 can list one or more subjective inputs received from the patient. Subjective inputs can include, for example, patient satisfaction or overall feeling, whether the patient has experienced fever, chills or night sweats, whether the patient is using pain medicine, whether the patient is feeling any side-effects of the pain medicine, a subjective pain rating, a subjective time and/or duration of the pain, a subjective perception of stability of the joint being operated, whether or not the patient has fallen, whether or not the patient has needed assistance, or whether or not the patient is using stairs. The subjective input can include, for example, responses to yes or no questions and/or questions requesting a subjective quantitative rating (e.g., a scale from 1 to 10) from the patient. An image 450 can be included in the sample report 400 to give a practitioner monitoring the patient's case optical feedback of the progress of a patient's joint 454 (e.g., a knee) for visualization of a surgical incision. A fifth field 460 can include, for example, results of data analysis performed by the process 300 at step 330. The data can include maximum flexion of the joint, maximum extension of the joint, total excursions per hour of the joint or the patient and/or modal excursion of the joint. A graph 470 can graphically represent the data shown, for example, in the fifth field 460. A sixth field 480 can be generated with data collected from the device 100 (FIGS. 1A-1D) and analyzed by the process 300 at step 330 (FIG. 3) to determine one or more activities that the patient has performed during the measurement period. These activities can include, for example, whether the patient is lying, sitting, standing, walking, taking the stairs, exercising, biking, etc. The sixth field 480 can include the duration of each activity and/or the change of the duration or magnitude of activity relative to one or more previous measurements. A graph 490 can provide a graphical representation of each activity in relation to the total duration of the measurement.

FIG. 5 is a flow diagram of a process 500 of a method of analyzing data configured in accordance with an embodiment of the present technology. In some embodiments, the process 500 can comprise instructions stored, for example, on the memory 254 of the computer 250 (FIG. 2) that are executable by the one or more processors 252. In one embodiment, for example, the process 500 can be incorporated into one or more steps (e.g., step 330) of the process 300 (FIG. 3). In certain embodiments, the process 500 comprises one or more techniques described by Rakthanmanon et al. in “Fast Shapelets: A Scalable Algorithm for Discovering Time Series Shapelets,” published in the Proceedings of the 2013 SIAM International Conference on Data Mining, pp. 668-676, and incorporated by reference herein in its entirety.

The process 500 starts at step 510. At step 520, the process 500 receives time series data from one or more sensors (e.g., data from the first and second sensor components 213c and 213d of FIG. 2 stored on the memory 254).

At step 530, the process 500 reduces the dimensionality of, or otherwise simplifies, the time series data received at step 520. In some embodiments, step 530 can include, for example, applying a Piecewise Linear Approximation (PLA) and/or a Piecewise Aggregate Approximation (PAA) to the data from step 520. In other embodiments, step 530 can include a decimation of the data from step 520. In further embodiments, however, any suitable technique for reducing dimensionality of time series data may be used such as, for example, Discrete Fourier Transformation (DFT), Discrete Wavelet Transformation (DWT), Single Value Decomposition (SVD) and/or peak and valley detection.

At step 540, the process 500 transforms the dimensionally reduced or otherwise simplified data of step 530 to a discrete space. Step 540 can include, for example, transforming the data of step 530 using Symbolic Aggregate approXimation (SAX). As those of ordinary skill in the art will appreciate, SAX is a technique by which data can be discretized into segments of a predetermined length and then grouped into two or more classes based on the mean value of the magnitude of the segment. Individual classes can be assigned letter names (e.g., a, b, c, d, etc.) and SAX words can be formed from the data, which can be used to classify the data.

At step 550, the process 500 detects one or more shapes or patterns in the discrete space data of step 540. At step 560, the process 500 matches the shapes and/or patterns detected at step 550 to a baseline data or learning data set, which can include, for example, one or more shaplets. The learning data set can be formed from data acquired from patients at various stages of recovery from a surgery and/or with various levels of ability can also be used to provide movement and/or activity recognition. The learning data set can comprise data from one or more individuals using the same sensor or group of sensors while performing the movement. The learning data set can be constructed, for example, using a machine learning algorithm comprising neural networks and/or classification trees configured to recognize activities or movements being performed by a patient. The process 500 can use the learning data to recognize movements in the data from step 550. Recognizable movements can include, for example, standing, lying on the left or right sides or the back or front with various combinations of joint flexion, extension, abduction, adduction, internal or external rotation, valgus or varus; sitting; seated with similar joint postures to those mentioned above; moving a joint while standing (e.g. standing knee flexion); cycling on a vertical bike; cycling on recumbent bike; exercising on an elliptical machine; running; walking; walking up stairs; walking down stairs; performing various therapeutic exercises; and sleeping. At step 570, the process 500 ends (e.g., returns to step 330 of FIG. 3).

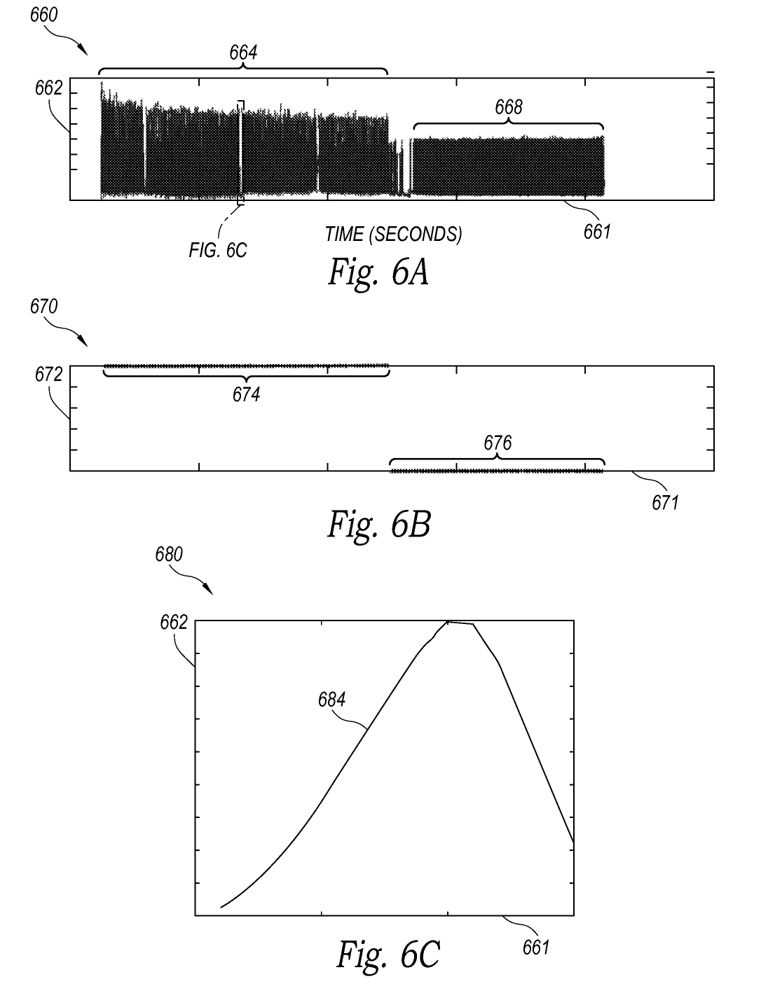

FIG. 6A is a graph 660 of data collected in accordance with an embodiment of the present technology. FIG. 6B is a discrete space graph 670 of the data of FIG. 6A after processing (e.g., by the process 500 of FIG. 5). FIG. 6C is a graph 680 of a portion of the data shown graph 660 of FIG. 6A. Referring first to FIGS. 6A and 6C, the graph 660 includes a first axis 661 (e.g., corresponding to time) and a second axis 662, which corresponds to a quantity (e.g., joint angle, joint angular velocity, joint acceleration, etc.) measured by a sensor in a device positioned proximate a patient's joint (e.g., the device 100 of FIGS. 1A-1D). A first data set 664 corresponds to measurement data acquired during a first period of time (e.g., a period of time lasting 20 minutes), and a second data set 668 corresponds to measurement data acquired during a second period of time (e.g., a period of time lasting 20 minutes). The graph 680 of FIG. 6C includes a shape, pattern or shapelet 684 from FIG. 6A that shows a shapelet that has previously been determined to characterize the sensor response pattern when the subject is performing a certain activity. For example, the shapelet 684 may have a shape or pattern that generally corresponds to the movement of a patient's knee as the patient climbs stairs. When the shapelet is compared to the data in data set 664 in FIG. 6A, a determination can be made regarding whether the subject was performing the activity represented by the shapelet. Another shapelet, from a library of shapelets, can be similarly applied to predict the activity being performed in the second data set 668. Referring next to FIG. 6B, the graph 670 includes a first axis 671 (e.g., corresponding to time) and a second axis 672 corresponding to, for example, activities (e.g., walking, climbing stairs, running, biking, etc.) performed by the patient and/or states (e.g., lying, sleeping, etc.) that the patient experiences during the measurement of the first and second data sets 664 and 668 of FIG. 6A. Data set 674 is a discrete transformation of the first data set 664 of FIG. 6A and classified as corresponding to a first activity (e.g., climbing stairs). Data set 676 is a discrete transformation of the first data set 664 of FIG. 6A and classified as corresponding to a second patient activity (e.g., walking)

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. The various embodiments described herein may also be combined to provide further embodiments.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Where the context permits, singular or plural terms may also include the plural or singular term, respectively. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/we claim:

1. A patient activity monitoring device, the device comprising:
a first body and a second body, wherein the first and second bodies are configured to be positioned proximate a joint of a human patient;
a flexible, elongate member extending from the first body toward the second body;
a first sensor disposed in the first body, wherein the first sensor is configured to acquire data indicative of motion of the patient;
a second sensor extending through the elongate member from the first body toward the second body, wherein the second sensor is configured to acquire data indicative of a flexion of the joint of the patient; and
a transmitter coupled to the first and second sensors, wherein the transmitter is configured to wirelessly transmit the data acquired from the first and second sensors to a computer.

2. The device of claim 1 wherein the computer is housed in a mobile device, and wherein the mobile device is configured to receive touch input from the patient, and wherein the mobile device is further configured to transmit the acquired data from the first and second sensors and the touch input data to a remote server communicatively coupled to a medical information system.

3. The device of claim 1 wherein the first sensor includes one or more accelerometers, and wherein the second sensor includes a goniometer.

4. The device of claim 1 wherein the first body, the second body and the elongate member are integrated into an article of clothing.

5. The device of claim 1 wherein the elongate member is configured to have a stiffness substantially less than a stiffness of the patient's joint.

6. The device of claim 1, further comprising
a control surface configured to receive touch input from the patient; and
one or more visual indicators.

7. The device of claim 1, further comprising one or more microphones configured to receive audio input from the patient.

8. A system for monitoring a patient, the system comprising:
a receiver configured to receive data indicative of motion of a joint, wherein the data is acquired by a sensor positionable on the patient proximate the joint;
memory configured to store the acquired data and executable instructions; and
one or more processors coupled to the memory and the receiver, wherein the one or more processors are configured to execute the instructions stored on the memory, and wherein the instructions include instructions for—
detecting one or more patterns in the acquired data;
determining one or more patient activities based on the one or more detected patterns; and
automatically generating a report that includes a list of one or more of the patient activities occurring during a predetermined period of time.

9. The system of claim 8 wherein the receiver, the memory and the one or more processors are housed in a computer remote from the sensor.

10. The system of claim 8, further comprising a mobile device communicatively coupled to the sensor via a first communication link and communicatively coupled to the receiver via a second communication link, wherein the mobile device is configured to receive audio, video and touch input data from the patient, and wherein the mobile device is further configured to transmit the data acquired by the sensor and the patient input data to the receiver via the second communication link.

11. The system of claim 10 wherein the generated report includes at least a portion of the patient input data.

12. The system of claim 8, further comprising a transmitter, wherein the transmitter and the receiver are configured to communicate with a medical information system via a communication link, wherein the instructions stored on the memory further include instructions for transmitting the generated report to the medical information system.

13. The system of claim 12 wherein the instructions stored on the memory further include instructions for triggering the scheduling of an appointment for the patient in the medical information system, wherein in the triggering is based on one or more of the patterns detected in the acquired data.

14. A method of assessing a function of a joint of a patient after a surgery performed on the joint, the method comprising:
receiving data from a sensor positioned proximate the patient's joint, wherein the sensor is configured to acquire data corresponding to an actuation of the patient's joint;
detecting one or more patterns in the acquired data;
determining one or more patient activities based on the one or more patterns detected in the acquired data; and
automatically generating a report that includes a list of each of the one or more of the patient activities.

15. The method of claim 14 wherein determining one or more patient activities includes comparing the one or more patterns detected in the acquired data with patterns in baseline data acquired from a different patient.

16. The method of claim 14 wherein detecting one or more patterns in the acquired data comprises reducing a number of dimensions in the acquired data from a first number of dimensions to a second, lower number of dimensions.

17. The method of claim 14 wherein detecting one or more patterns in the acquired data comprises applying shapelets to the data that are mathematically representative of one or more patient activities.

18. The method of claim 14, further comprising transmitting the generated report to a medical information system.

19. The method of claim 14, further comprising automatically scheduling an appointment based on one or more of the patterns detected in the acquired data.

20. The method of claim 14, further comprising automatically transmitting an alert to a health care practitioner based on information in the acquired data.
